(19) United States
(12) Patent Application Publication
(10) Pub. No.: US 2006/0039873 A1
(43) Pub. Date: Feb. 23, 2006

(54) Heavenly mouth wash

(76) Inventor: Annie McGee, Dallas, TX (US)

Correspondence Address: Annie McGee, 6850 S. Cockrell Hill #1514, Dallas, TX, 75236, US

(21) Appl. No.: 10/784,602

(22) Filed: Aug. 23, 2004

(51) Int. Cl. A61K 8/18

(52) U.S. Cl. 424/49

(57) ABSTRACT

Heavenly mouthwash has three natural ingredients. The first is one-third cup apple-cider vinegar, next is two-thirds cup water and last is one tablespoon grated lemon rind. When using the Heavenly mouthwash, you should first gargle with mouthwash for one minute to loosen scum on teeth and tongue, then use tongue scraper to remove scum from the tongue and finally brush teeth and rinse with water.

⅓ cup apple cider vinegar
⅔ cup water
1 tablespoon grated lemon rind

1. Gargle with mouthwash for 1 minute to loosen scum on teeth and tongue

2. Use tongue scraper to remove scum from the tongue

3. Brush teeth

4. Rinse with water

1. Heavenly mouthwash is an all natural product that includes no chemicals. It can be used as a tongue cleaner. It fights bad breath, while preventing and reduces plaque. It helps to loosen and remove scum on tongue, teeth and entire mouth. It removes particles between the teeth and makes mouth feel clean, while lessening visits to the dentist.
